(19) United States
(12) Patent Application Publication
(10) Pub. No.: US 2008/0153908 A1
(43) Pub. Date: Jun. 26, 2008

(54) Method of Treating Mucin Deficiency with an Active Pharmaceutical and Related Composition

(76) Inventor: Dharmendra M. Jani, Fairport, NY (US)

Correspondence Address: Bausch & Lomb Incorporated, One Bausch & Lomb Place, Rochester, NY, 14604-2701, US

(21) Appl. No.: 11/933,496

(22) Filed: Nov. 1, 2007

Related U.S. Application Data
(60) Provisional application No. 60/871,007, filed on Dec. 20, 2006.

(51) Int. Cl. A61K 31/19; A61P 27/02

(52) U.S. Cl. 514/557

(57) ABSTRACT

The present invention includes a method of treating a patient suffering from mucin deficiency comprising administering to an eye of a patient suffering from mucin deficiency, a composition comprising an active pharmaceutical selected from the group consisting of acetate, propionate or butyrate in their salt or acid forms and combinations thereof, wherein the active pharmaceutical is present in an amount effective to increase production of mucin in the eye of the mucin deficient patient.

### CROSS-REFERENCE

This application claims the benefit of Provisional Patent Application No. 60/871,007 filed Dec. 20, 2006, which is incorporated by reference herein.

### FIELD OF THE INVENTION

This invention relates to a composition for increasing the production of mucin in the eye and a related method of use and method of manufacture. In particular, the invention relates to a method of patients that have a mucin deficiency.

### BACKGROUND

The National Eye Institute/Industry Workshop (1998) defined dry eye as a disease that arises either because of decreased tear production or increased evaporation of tears that results in symptoms of ocular irritation. Recent estimates indicate that 10% to 30% of the adult population suffers from dry eye disease, with the prevalence increasing in older populations. Dry eye is caused by one of three types of deficiencies, mucin deficiency, lipid deficiency and aqueous tear deficiency.

Mucin deficiency occurs due to a failure of goblet cells and/or ocular surface epithelial cells to produce tear mucin. Deficiency of tear mucin destabilizes the tear film. Stevens-Johnson syndrome, burns and pemphigoid are the common causes of mucin deficiency. In the developing world, vitamin A deficiency (xerophthalmia) and trachoma are the most important conditions that affect the mucin layer of the tear film.

Lipid deficiency occurs when the meibomian glands fails to produce anormal amount of lipid. Lipids produced from the meibomian gland contributes to an anterior oily layer of the tear film. The oily layer prevents evaporation of the tear film. The most common causes of tear lipid deficiency include blepharitis and meibomitis. Radiation therapy can cause meibomian gland dropout, leading to a serious deficiency in the tear lipid layer.

Aqueous tear deficiency occurs when the lacrimal gland fails to produce the aqueous portion of the tears. The aqueous layer of the tear film lies in between the lipid and mucin layers and forms the bulk of the tear film. The aqueous layer also dissolves tear mucins, making it more of a gel-like layer.

Dry eye conditions are often treated with a generally aqueous formulation to restore fluid to the eye. A humectant is present in the formulation to assist in the retention of water. Humecants include non-polymeric polyols because of their lubricious nature and ability to retain water. Polymeric humectants such as hydroxypropylmethylcellulose, carboxymethylcellulose, hyaluronic acid, polyacrylic acid and alginate are useful because they increase the viscosity of the formulation. As a result the resident time is improved.

U.S. Patent No. 2004-0014812 discloses that farnesyl acetic acid is useful in an ophthalmic solution to stimulate mucin production.

U.S. Pat. No. 6,271,216 discloses a hyaluronate viscoelastic formulation that was made with a balanced salt solution. Acetate salts including calcium acetate, magnesium acetate potassium acetate and sodium acetate were compositions that were added to provide the necessary amount of calcium, magnesium potassium and sodium ions in solution.

GB Patent No. 1320316A states that acetic acid is an acceptable pH adjusting agent.

JP7017863A discloses a formulation that reduces the irritation caused by the active ingredient pranoprofen. Acetate ions are added in the form of acetic acid and/or sodium acetate.

Finnie, et al., “Colonic mucin synthesis is increased by sodium butyrate,” Gut, vol. 36 (1), pp. 93-99 (1995) teaches that about 0.1 millimolar concentration of sodium butyrate used to harvest tissue from colonic cancer patients caused an almost five fold increase in colonic mucin production by the harvested tissue compared to a control group.

Barcello, et al., “Mucin secretion is modulated by luminal factors in the isolated vascularly profused rat colon,” Gut, vol. 46, pp. 218-224 (2000) shows that acetate, propionate and butyrate can stimulate the mucin secretion of rat colon cells.

In view of the above, it would be desirable to provide an eye-drop solution that will stimulate mucin production in the eye of a patient that has mucin deficiency and that is safe, convenient and economical to use. The present invention addresses these and other needs.

### SUMMARY OF THE INVENTION

The present method is directed to treating mucin deficiency in the eye. The method comprises administering an active pharmaceutical to the eye of a patient suffering from mucin deficiency. The composition comprises an active pharmaceutical selected from the group consisting of acetate, propionate and butyrate in their salt or free acid forms and combinations thereof. The active pharmaceutical is present in an amount sufficient to increase the mucin production in a patient.

A patient with mucin deficiency is a patient that has less than a normal amount of mucin. In one embodiment, the mucin deficient patient produces one natural log order less mucin than does the average population.

In one embodiment, the viscosity is adjusted to a maximum of about 30000 cps.

In another embodiment, the composition further comprises a polymeric viscosifier that is selected from the group consisting of carbomer, carboxymethylcellulose, hydroxypropylmethylcellulose, ethylcellulose, hyaluronic acid, chondroitin sulfate, alginate, agar, guar, xanthan gum or poly(vinyl alcohol) in their salt, base or acid forms and combinations thereof. In one embodiment, the composition further comprises alginate.

The concentration of alginate is, preferably, a minimum of about 0.01 wt. % and a maximum of about 5 wt. % based upon the total weight of the composition.

In another embodiment, the buffer(s) are selected from the group comprising phosphate buffer, borate buffer, MOPS buffer, citrate buffer, an aminoalcohol buffer and combinations thereof including but not limited to a phosphate/borate buffer and a citrate/borate buffer.

In still another embodiment, the pH of the composition is a minimum of about 4 and a maximum of about 8.

In one embodiment, the tonicity of the composition is a minimum of about 200 and a maximum of about 400 mOsm/kg.

In another embodiment the method results in no less than a ½ natural log order increase in mucin production.

In an embodiment, the compound is butyrate in its acid or salt form.

In another embodiment, there is a composition for treatment of mucin deficiency comprising an aqueous solution comprising an active pharmaceutical selected from the group consisting of acetate, propionate, butyrate in their acid or salt forms and combinations thereof, wherein the active pharmaceutical is present in an amount effective to increase mucin production.

In still another embodiment, there is a use of an active pharmaceutical selected from the group consisting of butyrate, propionate, and acetate in their salt or acid forms and combinations thereof in the manufacture of a medicament for treatment of mucin deficiency in an amount effective to increase mucin production in the eye of a mucin deficient patient.

### DETAILED DESCRIPTION OF THE INVENTION

The present composition may also contain a disinfecting amount or a preserving amount of an antimicrobial agent. Antimicrobial agents are defined as chemicals that derive their antimicrobial activity through a chemical or physiochemical interaction with the microbial organisms. These include sorbic acid, quaternary ammonium polymers and low and high molecular weight biguanides. For example, biguanides include the free bases or salts of alexidine, chlorhexidine, hexamethylene biguanides and their polymers, and combinations of the foregoing. The salts of alexidine and chlorhexidine can be either organic or inorganic and are typically gluconates, nitrates, acetates, phosphates, sulfates, halides and the like. A preferred polymeric biguanide is poly(hexamethylene biguanide) commercially available from Zeneca, Wilmington, Del. under the trademark Cosmocil™ CQ. Generally, the hexamethylene biguanide polymers, also referred to as poly(aminopropyl biguanide) (PAPB), have molecular weights of up to about 100 kDa. A particularly preferred preservative is alexidine.

If used in the subject solution, the antimicrobial agent should be used in an amount which will preserve or prevent the growth of the microorganism population in the formulations employed. Preferably, a preservative amount is that which will reduce the bacterial bioburden after 28 days each by 3 logs and prevents the growth of fungal bioburden by ±0.5 log. Typically, such agents are present in a minimum concentration of about 0.0001 wt. %, 0.0003 wt. % or 0.0005 wt. % and a maximum concentration of about 0.0005 wt. % or 0.001 wt. % or about 0.005 wt. % based upon the total weight of the composition.

The composition optionally contains a viscosifier to increase the residence time of the vehicle in the eye of a patient. The viscosifier is typically an ophthalmically safe polymer. Examples of soluble, ophthalmically safe polymers include but are not limited to carbomer, carboxymethylcellulose, hydroxypropylmethylcellulose, ethylcellulose, hyaluronic acid, chondroitin sulfate, alginate, agar, guar, xanthan gum or poly(vinyl alcohol). The viscosifiers also retain water and improve comfort. A preferred viscosifier is alginate.

Non-polymeric humectants are optionally added to the composition of the present invention. A non-polymeric humectant is a chemical that holds or retains water and is capable of providing moisture to the surface of the eye. Polyols are a non-limiting example of a non-polymeric humectant. The polyol of one embodiment of the present invention is typically contains 2 to 6 carbon atoms. Preferably, the polyol contains 2 to 4 carbon atoms. The polyol of one embodiment is selected from the group consisting of glycerin, ethylene glycol, poly(ethylene glycol), propylene glycol, sorbitol, manitol and monosaccarides, disaccharides, oligosaccharides and neutral polysaccharide. In one preferred embodiment, the polyol is selected from the group consisting of glycerin, ethylene glycol, propylene glycol, sorbitol, manitol and monosaccharides. In another preferred embodiment, the polyol is selected from the group comprising disaccharides, oligosaccharides and poly(ethylene glycol). In one preferred embodiment, the polyol is glycerin.

The concentration of polyol including glycerin is a minimum of about 0.01 wt. % about 0.05 wt. % about 0.1 wt. % or about 0.5 wt. %, about 1 wt. %, and/or a maximum of about 1.5 wt. %, about 2 wt. %, about 3 wt. %, about 4 wt. % or about 5 wt. % based upon the total weight of the composition.

The aqueous solutions employed in this invention may contain additional ingredients described above, one or more other components that are commonly present in ophthalmic solutions, for example, buffers, stabilizers, tonicity agents and the like, which aid in making ophthalmic compositions more comfortable to the user. The aqueous solutions of the present invention are typically adjusted with tonicity agents to approximate the tonicity of normal lacrimal fluids which is equivalent to a 0.9 wt. % solution of sodium chloride or a 2.8 wt. % of glycerol solution. The solutions are made substantially isotonic with physiological saline used alone or in combination; otherwise, if simply blended with sterile water and made hypotonic or made hypertonic, the lenses will lose their desirable optical parameters. Correspondingly, excess salt or other tonicity agents may result in the formation of a hypertonic solution that will cause stinging and eye irritation. An osmolality is a minimum of about 200 mOsm/kg, about 225 mOsm/kg, about 250 mOsm/kg, about 260 mOsm/kg, about 280 mOsm/kg, about 300 mOsm/kg or about 320 mOsm/kg and/or a maximum of about 400 mOsm/kg, about 380 mOsm/kg, about 360 mOsm/kg, about 340 mOsm/kg or about 320 mOsm/kg. Most preferably, the osmolality is about 240 mOsm/kg to about 320 mOsm/kg.

Preferably, the composition of at least one embodiment of the present invention has a low ionic strength. Typically, the composition contains low concentration of mono or divalent cations typically found in tear fluids. Generally, the composition contains a low concentration of one or more of the following cations: Na+, K+, Ca++, Mg++, and Zn++. In one embodiment, the concentration of the mono or divalent cations that are typically found in tear fluids (i.e. Na+, K+, Ca++, Mg++and Zn++) has a minimum concentration of about 0.001 wt. %, about 0.005 wt. %, about 0.01 wt. % or about 0.1 wt. % and/or a maximum of about 0.1 wt. %, about 0.01 wt. %, about 0.1 wt. %, about 0.05 wt. % or about 0.01 wt. % based upon the total weight of the composition.

The pH of the present composition should be maintained at a minimum of about 4 about 5, about 5.5, about 6, about 6.5 and/or a maximum of about 7.5, about 7.8, about 8, about 8.5. Suitable buffers may be added, such as borate, citrate, bicarbonate, aminoalcohol buffers, MOPS buffer, bicine, tricine, TRIS, BIS/TRIS and various mixed phosphate buffers (including combinations of Na₂HPO₄, NaH₂PO₄ and KH₂PO₄) and mixtures thereof. Borate buffers are preferred, particularly for enhancing the efficacy of PAPB. Preferred combination buffers include borate/phosphate and borate/citrate combination buffers. Generally, buffers will be used in amounts having a minimum of about 0.05 wt. % or about 0.1 wt. % and/or a maximum of about 1.5 wt. % or about 2.5 wt. %.

In addition to buffering agents, in some instances it may be desirable to include sequestering agents in the present solutions in order to bind metal ions, which might otherwise react with the lens and/or protein deposits and collect on the lens. Ethylene-diaminetetraacetic acid (EDTA) and its salts (disodium) are preferred examples. They are usually added in amounts having a minimum of about 0.01 wt. % and/or a maximum of about 0.2 wt. %.

In one embodiment, there is a method of manufacturing a composition for treatment of mucin deficiency. The method of manufacturing comprises adding to an aqueous solution, ophthalmically pure alginate. As indicated above, the present invention is useful for treating mucin deficiency. For that purpose, compositions for use in the present invention may be sold in a wide range of small-volume containers from 1 ml to 30 ml in size. Such containers can be made from HDPE (high density polyethylene), LDPE (low density polyethylene), polypropylene, poly(ethylene terepthalate) and the like. Flexible bottles having conventional eye-drop dispensing tops are especially suitable for use with the present invention.

The above-described solutions, in accordance with the present invention, may be used by instilling, for example, about one (1) or three (3) drops in the affected eye(s) as needed to increase the level of mucin in the eye.

### EXAMPLE 1

### Formulation

The following ingredients and respective amounts are used to make a base formulation:

| | Minimum | Maximum | Exemplary |
|---|---|---|---|
| | Amount | Amount | Amount |
| | (% w/w) | (% w/w) | (% w/w) |
| Formulation 1 | | | |
| Boric Acid | 0.05 | 2 | 0.5 |
| Sodium Borate | 0.005 | 0.5 | 0.014 |
| Glycerin | 0.1 | 5 | 0.6 |
| Propylene Glycol | 0.1 | 5 | 0.6 |
| Sodium Butyrate | 0.1 | 2 | 0.5 |
| HAP (30%) | 0.05 | 1 | 0.5 |
| Alexidine 2HCl | 1 ppm | 5 ppm | 3 ppm |
| Purified Water | Q.S. to 100 | Q.S. to 100 | Q.S. to 100 |
| Formulation 2 | | | |
| Boric Acid | 0.05 | 2 | 0.5 |
| Sodium Borate | 0.005 | 0.5 | 0.014 |
| Glycerin | 0.1 | 5 | 0.6 |
| Propylene Glycol | 0.1 | 5 | 0.6 |
| Sodium Alginate | 0.05 | 2 | 0.25 |
| Sodium Butyrate | 0.1 | 2 | 0.25 |
| HAP (30%) | 0.05 | 1 | 0.5 |
| Alexidine 2HCl | 1 ppm | 5 ppm | 3 ppm |
| Purified Water | Q.S. to 100 | Q.S. to 100 | Q.S. to 1000 mg |

### Formulation Process:

The formulations of the present invention are made as follows: All the ingredients are weighed as per the formulation. Mix the dry ingredients (boric acid, sodium borate, sodium butyrate, sodium alginate) together. Weigh 90% of the total water required and add in all the liquid components (HAP, propylene glycol, glycerin). Then gradually add the dry blended powder mix. Warm the solution to no more than 45 C to accelerate the dissolution process for the polymers and dry powders. Add in the antimicrobial, eg. alexidine (preferably from a stock solution) to the desired concentration. Sterile filter the final mix through a 0.2 μm filter and store in a clean container.

### EXAMPLE 2

### Stimulation of Mucin Production

A 0.5 wt. % solution of sodium butyrate in artificial tear solution is administered to one of a group of two patients that suffer from mucin deficiency. The patients receiving butyrate drops belong to the study group. The patients in the control group receive artificial tear solution. Both groups receive treatment four-times a day for four days. Following treatment, tear film samples are collected and tested to quantify mucin content. The patients in the test group are expected to have a higher concentration of mucin than the patients in the control group. This shows that butyrate drops increase the production of mucin.

While the invention has been described in conjunction with the detailed description and specific examples, this is illustrative only. Accordingly, many alternatives, modifications and variations will be apparent to those skilled in the art in light of the foregoing description and it is, therefore, intended to embrace all such alternatives, modifications and variations as to fall within the spirit and scope of the appended claims.

What is claimed is:

1. A method of treating mucin deficiency in the eye, the method comprises administering to an eye of a patient suffering from mucin deficiency, a composition comprising an active pharmaceutical selected from the group consisting of acetate, propionate and butyrate in their salt or free acid forms and combinations thereof, wherein the active pharmaceutical is present in an amount sufficient to increase the mucin production in a patient.

2. The method of claim 1, where in the patient, without treatment, produces one natural log order less mucin than does the average population.

3. The method of claim 1, wherein the composition further comprises a polymeric viscosifier that is selected from the group consisting of carbomer, carboxymethylcellulose, hydroxypropylmethylcellulose, ethylcellulose, hyaluronic acid, chondroitin sulfate, alginate, agar, guar, xanthan gum or poly(vinyl alcohol) in their salt, base or acid forms and combinations thereof.

4. The method of claim 1, wherein the composition further comprises alginate.

5. The method of claim 4, wherein the concentration of alginate is a minimum of about 0.01 wt. % and a maximum of about 5 wt. % based upon the total weight of the composition.

6. The method of claim 1, wherein the tonicity of the composition is a minimum of about 200 and a maximum of about 400 mOsm/kg.

7. The method of claim 1, wherein the method results in no less than a ½ natural log order increase in mucin production.

8. The method of claim 1, wherein the compound is butyrate in its acid or salt form.

9. A composition for treatment of mucin deficiency comprising an aqueous solution comprising an active pharmaceutical selected from the group consisting of acetate, propionate, butyrate in their acid or salt forms and combinations thereof, wherein the active pharmaceutical is present in an amount effective to increase mucin production in the eye of a subject receiving said composition.

10. The composition of claim 9, wherein the active pharmaceutical is butyrate in its salt or acid form.

11. The composition of claim 10, wherein the composition further comprises an active pharmaceutical is in an ophthalmically acceptable vehicle.

12. The composition of claim 11, wherein the composition has a viscosity that is a maximum of about 30000 cps.

13. The composition of claim 9, wherein the composition further comprises a polymeric viscosifier that is selected from the group consisting of carbomer, carboxymethylcellulose, hydroxypropylmethylcellulose, ethylcellulose, hyaluronic acid, chondroitin sulfate, alginate, agar, guar, xanthan gum or poly(vinyl alcohol) in their salt, base or acid forms and combinations thereof.

14. The composition of claim 9, wherein the composition further comprises alginate.

15. The composition of claim 14, wherein the concentration of alginate is a minimum of about 0.01 wt. % and a maximum of about 5 wt. % based upon the total weight of the composition.

16. The composition of claim 9, wherein the composition further comprises a buffer selected from the group comprising phosphate buffer, borate buffer, MOPS buffer, citrate buffer, an aminoalcohol buffer and combinations thereof including but not limited to a phosphate/borate buffer and a citrate/borate buffer.

17. The composition of claim 9, wherein the pH of the composition is a minimum of about 4 and a maximum of about 8.

18. The composition of claim 9, wherein the tonicity of the composition is a minimum of about 200 and a maximum of about 400 mOsm/kg.

19. A method for manufacturing a composition for treating mucin deficiency in a subject, the method comprising combining (a) an active pharmaceutical selected from the group consisting of butyrate, propionate, and acetate, and combinations thereof, wherein each of said active pharmaceutical is in in salt or acid form; and (b) a pharmaceutically acceptable carrier, to form said composition.

20. The method of claim 19, further comprising adjusting a tonicity of the composition to a value in a range from about 200 to about 400 mOsm/kg.
